(19) United States
(12) Patent Application Publication
(10) Pub. No.: US 2009/0170816 A1
(43) Pub. Date: Jul. 2, 2009

(54) COMBINATION COMPRISING SQUALENE, A PHOSPHOLIPID AND AN OMEGA 3 FATTY ACID FOR THE TREATMENT OF CANCER

(75) Inventors: Aida Lacasa Mas, Barcelona (ES); Diana Lacasa Mas, Barcelona (ES); Joan Cunill Aixela, Barcelona (ES)

Correspondence Address: SUGHRUE MION, PLLC, 2100 PENNSYLVANIA AVENUE, N.W., SUITE 800, WASHINGTON, DC, 20037, US

(73) Assignee: FARMALEIS S.L., BARCELONA (ES)

(21) Appl. No.: 12/094,082

(22) PCT Filed: Oct. 25, 2006
(86) PCT No.: PCT/EP2006/010276

§ 371 Date: Jul. 25, 2008

(30) Foreign Application Priority Data
Nov. 18, 2005 (ES) P 200502835

(51) Int. Cl. A61K 31/66

(52) U.S. Cl. 514/121

(57) ABSTRACT

Pharmaceutical composition which comprises squalene or its pharmaceutically acceptable derivatives; at least one phospholipid, its salts or pharmaceutically acceptable derivatives; and at least one omega-3 fatty acid, its salts or pharmaceutically acceptable derivatives. Use of the composition in the preparation of a drug designed for the prophylaxis, treatment and/or improvement of cancer symptoms in mammals. The invention also relates to a fodder for animals such as dogs and cats which comprises the composition.

### TECHNICAL FIELD OF THE INVENTION

This invention relates to a pharmaceutical composition useful in the prophylaxis or treatment of cancer in animals, particularly mammals and, more specifically, domestic animals such as dogs and cats.

The invention also relates to a fodder for domestic animals, especially for dogs and cats.

### BACKGROUND OF THE INVENTION

Cancer may be defined, in a generic manner, as the uncontrolled growth of cells that have suffered some type of mutation. Currently, cancer is one the most frequent causes of death in domestic animals, especially in dogs and cats. Death of the animal results from the fact that the above-mentioned abnormal cells end up preventing the correct functioning of affected vital organs, or that said cells, due to their invasive capacity or to metastasis, end up affecting other essential systems.

In general, cancer has multiple, varied causes, and there are many types of cancers. In any event, what they all have in common is that an alteration in the regulation of cell division takes place in a given tissue, leading to uncontrolled cellular proliferation that agglomerates in the form of tumours, or else spreads through the bloodstream, which may lead to the transformation of normal cells into abnormal cells in tissues far removed from the first cancer focus. This deregulation is usually the result of errors or alterations in the cells' DNA, which in turn are the result of spontaneous genetic mutations induced by external stimuli or by hereditary factors.

There are three types of genes involved in the development of cancer. The first group is that of DNA-repair genes, which are responsible for correcting the errors produced during DNA replication. The second group of genes is that of oncogenes, which are genes that participate in mitogenesis. Finally, there are tumour-suppressing genes, which control cell division, restraining it when a high rate of cellular proliferation is not necessary. Unbalance in the regulation and simultaneous action of all these genes leads to the appearance of cancer.

Currently, the therapies used consist of surgically removing the tumour, the application of radiation in the affected areas and/or chemotherapy, or the administration of drugs which eliminate the malignant cells or tumours.

In domestic animals, such as dogs and cats, removal of the tumour is usually performed if the tumour is localised, combining it, if necessary, with radiotherapy and chemotherapy sessions. These radiotherapy sessions, in addition to being costly and very unpleasant for the animals, have the disadvantage that the owner of the animal must frequently visit the veterinary centre, which often leads to the decision to put the animal to sleep.

Chemotherapy is currently in a sufficiently advanced stage so that it may be performed at the animal owner's home, for example, by the administration of oral formulations. However, the compositions that are administered usually have unpleasant organoleptic properties that may cause the animal to regurgitate them or refuse to swallow them, which sometimes leads to them also having to be administered by a veterinarian.

Currently, studies are being conducted on the anticarcinogenic or reparative properties of multiple natural compounds, of virtually daily use in diets. One example of this is squalene. Squalene is a triterpene compound, which is used as an intermediate in the biosynthetic pathway of endogenous cholesterol and of steroids. Squalene is found in high proportions in olive oil.

Squalene has turned out to be very beneficial in the treatment of different types of cancer. One example is that described in “Inhibition of 4-(methylnitrosamino)-1-(3pyridyl)-1-butanone-induced lung tumorigenesis by dietary olive oil and squalene” by Smith, T et al, Carcinogenesis vol. 19 no.4 pp. 703-706, 1998. Smith et al. induce lung tumours in rats and, subsequently, they administer squalene, or squalene in olive oil, to them. One can observe that the animals who received squalene exhibit a lower incidence of tumours, as well as a lower multiplicity and smaller volume thereof, as compared to those who did not. Said study shows that olive oil or the squalene contained therein acts as a potent inhibitor of the genesis of chemically-induced lung tumours. It is hypothesised that a possible action mechanism is due to the inhibition of the farnesylation of Ras proteins, by inhibition of 3-hydroxy-3-methylglutaryl-coenzyme A (HMG CoA) by squalene.

Another property of squalene that explains that it is a good anticarcinogenic agent is that it is potent as an antioxidant and in capturing free radicals, thus preventing damage to the cells' molecules (DNA, lipids or proteins) which may lead to cell undifferentiation processes, or cancer.

There are also patents which disclose compositions comprising squalene. For example, JP 56113794 discloses a composition with squalene in the form of thymidinephenoxyacetate ester. The composition is useful in preventing or inhibiting the metastasis of invasive-type cancers.

JP 61148119 also discloses a composition with squalene and nimustine hydrochloride, which also has inhibitory effects on the metastasis of hematopoietic system cancers, such as fibrosarcomas.

Another natural product that seems to have anticarcinogenic properties are unsaturated fatty acids of the omega-3 type. An omega-3 fatty acid is understood to be a fatty acid which has an unsaturated bond located three carbons away from the omega carbon. One example of the use of omega-3 fatty acids in fighting cancer is that disclosed in WO2005/034874, which shows a therapeutic method that consists of administering an adequate quantity of an omega-3 fatty acid associated with a hydrophobic carrier, of general use in cancer therapy, to a cancer patient. However, this patent application does not show significant efficacy data for the therapeutic method.

Phospholipids are also being used in anti-cancer therapies. Thus, US2005/0064025 discloses a composition with phospholipids and an anti-inflammatory drug which improves cancer symptoms. It presents trials which demonstrate that the composition is effective in bladder cancer and in a cell line from a mammary tumour.

However, from the state of the art one may deduce that there is a lack of effective compositions against cancer which, in addition, are pleasant and easy to be consumed by animals, especially by oral route.

The inventors have developed a composition which has surprising effects when used to manufacture an anti-cancer drug and, in addition, offers numerous advantages as compared to the compositions currently existing in the market.

### DESCRIPTION OF THE INVENTION

The pharmaceutical composition described in the invention is characterised in that it comprises squalene, or its pharmaceutically acceptable derivatives; at least one phospholipid, its salts, or pharmaceutically acceptable derivatives; and at least one omega-3 fatty acid, its salts or pharmaceutically acceptable derivatives.

Preferably, the pharmaceutical composition comprises squalene, at least one omega-3 fatty acid and at least one phospholipid in a weight proportion of 1:1:2.

According to another characteristic of the pharmaceutical composition according to the invention, squalene, at least one omega-3 fatty acid and at least one phospholipid are in a weight proportion of 3:5:10.

Preferably, the phospholipid is phosphatidylserine, phosphatidylcholine or a mixture of both.

Another object of the invention is the use of a composition that comprises squalene or its pharmaceutically acceptable derivatives; at least one phospholipid, its salts or pharmaceutically acceptable derivatives; and at least one omega-3 fatty acid, its salts or pharmaceutically acceptable derivatives, in the preparation of a drug designed for the prophylaxis, treatment and/or improvement of cancer symptoms in animals.

Preferably, the composition is used in the preparation of a drug designed for the prophylaxis, treatment and/or improvement of breast cancer in mammals.

According to another characteristic of the invention, the use of the composition is for the preparation of a drug designed for the prophylaxis, treatment and/or improvement of skin cancer in mammals, for the prophylaxis, treatment and/or improvement of digestive system cancer and genitourinary system cancer in mammals.

Preferably, it is expected that the composition be used in the preparation of a drug designed for oral administration.

According to another aspect of the invention, the pharmaceutical composition is used in the preparation of an injectable-formulation drug or for topical administration.

Another object of the invention is a fodder for animals which comprises a pharmaceutical composition with squalene or its pharmaceutically acceptable derivatives; with at least one phospholipid, its salts or pharmaceutically acceptable derivatives; and with at least one omega-3 fatty acid, its salts or pharmaceutically acceptable derivatives.

Another object of this invention is an oral administration pharmaceutical capsule which comprises squalene or its pharmaceutically acceptable derivatives; at least one phospholipid, its salts or pharmaceutically acceptable derivatives; and at least one omega-3 fatty acid, its salts or pharmaceutically acceptable derivatives, with squalene, the omega-3 fatty acid and the phospholipid in a weight proportion of 3:5:10.

Another object of this invention is an oral administration pharmaceutical suspension which comprises squalene or its pharmaceutically acceptable derivatives; at least one phospholipid, its salts or pharmaceutically acceptable derivatives; and at least one omega-3 fatty acid, its salts or pharmaceutically acceptable derivatives, with squalene, the omega-3 fatty acid and the phospholipid in a weight proportion of 1:1:2.

In this invention, when squalene is mentioned it refers to the triterpene, as well as the possible derivatives thereof, such as squalene oxide.

Phospholipid is understood to be any compound with a phosphate group bound to a diglyceride, to the salts of, for example, said phosphate group or the aminoesters of the phosphoric acid bound to fatty acid diglycerides, such as, for example, phosphatidylcholine.

Omega-3 fatty acid is understood to be any fatty acid which has an unsaturated bond located three carbons away from the omega carbon, whether in the form of a free acid or else combined to form esters, amides or salts, solvated or anhydrous; the omega-3 fatty acid may be either a monounsaturated fatty acid or a polyunsaturated fatty acid.

### BRIEF DESCRIPTION OF THE DRAWINGS

In order to better exemplify the results of the composition of the invention, the following figures are attached:

FIG. 1 is a pie chart with sections indicating the percentages of survival, death and relapses observed following administration of the composition according to the invention in animals suffering from skin or soft tissue cancer;

FIG. 2 shows the results upon administering the composition to dogs and cats suffering from digestive system cancers;

FIG. 3 is another pie chart with sections indicating the percentages of survival, death and relapses, observed following administration of the composition according to the invention in dogs and cats with different types of breast cancer; and

FIG. 4 shows the results of administering the pharmaceutical composition according to the invention in animals (dogs and cats) with cancer in any organ of the genitourinary system.

### DETAILED DESCRIPTION OF THE DRAWINGS

The object of this invention is a pharmaceutical composition which exhibits surprising effects when used, jointly with the suitable excipients, in the preparation of a drug designed for the treatment of certain types of cancer in mammals.

The composition comprises, as active principles which act in a synergic manner, squalene, at least one type of phospholipid and at least one type of omega-3 fatty acid. Said active principles may be used in free form or in the form of their pharmaceutically acceptable salts, as well as any other derivative, such as, for example, the esters or amines thereof.

The pharmaceutical composition preferably comprises squalene, at least one omega-3 fatty acid and at least one phospholipid in a weight ratio of 1:1:2. Another preferred weight ratio amongst the three components is 3:5:10.

### Example 1

### Pharmaceutical Composition According to the Invention

The following composition corresponds to a preferred embodiment. It is formulated as oral administration capsules.

| Compound | Weight in mg | |
|---|---|---|
| Squalene | 30 | mg |
| Omega-3 fatty acid | 50 | mg |
| (alpha-linolenic acid) | | |
| Phospholipids | 100 | mg |
| (Soy lecithin) | | |

| | |
|---|---|
| Excipients | q.s. total weight of the capsule (including |
| | the gelatins for formation of the capsule) |

The total weight of the capsule is 562 mg±0.01. A capsule with this composition is administered daily to animals with a weight less than 15 kg. If the animal's weight is greater than 15 kg, it is recommended to administer 2 capsules per day.

The squalene may be of animal origin, obtained from shark cartilage, or of vegetable origin, that is, obtained from olive oil.

In the case represented in this Example 1, the source of phospholipids is soy lecithin, which is rich in phosphatidylcholine. However, phosphatidylserine may be used as the phospholipid, and the same efficacy is obtained.

Similarly, although in the case of Example 1a vegetable fatty acid is used as the omega-3 fatty acid, it may perfectly well be replaced by eicosapentaenoic acid (EPA) and docosahexaenoic acid (DHA), both of animal origin.

The excipients used are all those known by persons skilled in the art, which are suitable to obtain the desired galenic form.

### Example 2

### Fodder Containing the Pharmaceutical Composition According to the Invention

In order to facilitate administration of the above-mentioned pharmaceutical composition with squalene, at least one phospholipid and at least one omega-3 fatty acid, it is foreseen that it be formulated as an additive in fodders for animals, especially fodder for dogs and cats.

The adequate quantity of the pharmaceutical composition according to the invention is added to a fodder of the type which usually comprises animal or vegetable proteins, vitamins and trace elements, as well as fatty matter, also of animal or vegetable origin, generally produced from excess materials resulting from human food industry processes, in order that the animals consume, depending on their weight, the recommended daily dose thereof. In general, 400 mg of the pharmaceutical composition are added per 1 kilogram of fodder.

A fodder such as the one described may also prevent the risk of cancer in domestic animals, prolonging their life and, more importantly, the quality of life of the animals and their owners, who will avoid recurrent visits to a veterinarian.

### Example 3

### Oral Suspension

The following composition corresponds to another preferred embodiment according to the invention. It is formulated as suspensions intended for oral administration.

| Compound | Weight in mg | |
|---|---|---|
| Squalene | 50 | mg |
| Omega-3 fatty acid | 50 | mg |
| Phospholipid | 100 | mg |

| | |
|---|---|
| Excipients | q.s. 100 grams of suspension |

In this Example 3, the omega-3 fatty acids, as well as the squalene, may also be of animal or vegetable origin. The phospholipids used are, without distinction, phosphatidylcholine or phosphatidylserine or a mixture of both.

The excipients used are all those known by persons skilled in the art, which are suitable to obtain the desired galenic form.

This galenic form is very advantageous because it is very easy to administer in the animals' mouth, for example by means of a syringe between the jaws.

Trials.

In order to exemplify the use of the pharmaceutical composition according to the invention, several trials were conducted in dogs and cats who had been diagnosed with some type of cancer. They were all administered one capsule per day in accordance with Example 1 if their weight was less than or equal to 15 kilograms and two capsules per day if the animals' weight was greater than 15 kilograms.

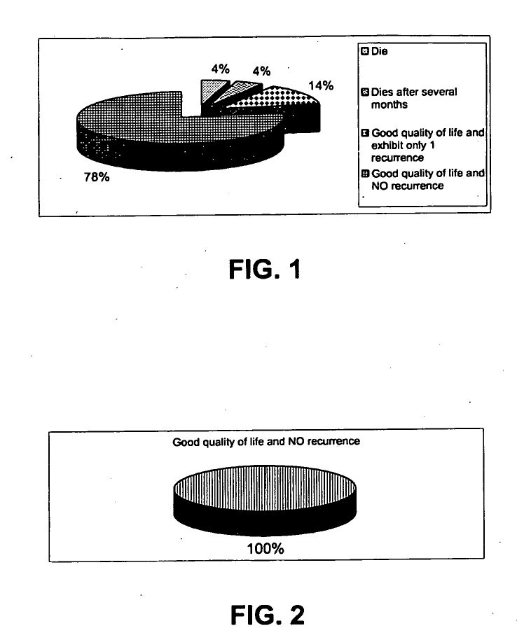

FIG. 1 shows a pie chart with the percentages of recovery, death and relapse of the animals treated with the composition of the invention, who had been diagnosed with some type of skin or soft tissue cancer, such as skin melanomas, mastocytomas, tumours in the apocrine sweat glands, pilomatricomas, lipomas, schwannomas, basaliomas, melanosarcomas, adenocarcinomas of the ceruminous glands, histiocytomas, fibrohistiocytomas, fibrosarcomas or histiocytic sarcomas.

Said FIG. 1 clearly shows that the mortality rate is only 8% (4% died after several months of administration and another 4% died soon because there was a metastasis with a high degree of invasion). The remaining 92% survived without recurrence (78%) and only 14% exhibited a slight relapse.

FIG. 2 shows that the administration of the capsules of example 1 led to 100% recovery and a good quality of life for dogs and cats who suffered from digestive system cancers, in the form of liver carcinomas, colorrectal papilomas, leiomyosarcomas or Epuli (periodontal fibrous hyperplasia).

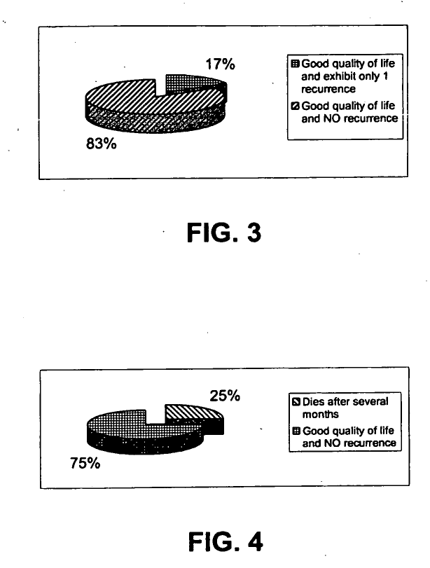

Finally, FIG. 3 is another pie chart with sectors showing the percentages of survival with good quality of life and without recurrences (83%) and with good quality of life and with a single recurrence (17%) in those animals (dogs and cats) who had been diagnosed with mammary tissue cancers.

It has also been observed that genitourinary system cancers have a good prognosis in dogs and cats if a drug prepared with the pharmaceutical composition according to the invention is administered thereto. An example thereof may be seen in the results of FIG. 4. They show the percentages of survival and death for animals diagnosed with urinary bladder carcinomas, ovarian mesotheliomas or sertolinomas. Although 25% of the treated animals died, one should highlight that the remaining 75% survived and that they did not exhibit any recurrence, in addition to having a good quality of life.

All these results show that, in fact, the pharmaceutical composition of the invention with squalene, at least one type of phospholipid and at least one type of omega-3 fatty acid is a combination that is effective in fighting cancer, especially breast cancer, skin cancer, genitourinary system cancer and digestive system cancer, especially liver cancer.

The effects of the pharmaceutical composition in hematopoietic system cancers, such as lymphomas, hemangiosarcomas or lymphosarcomas, have also been studied. In this type of cancers, survival rates of about 56% are achieved.

A situation similar to that of the hematopoietic system is that observed when applying the composition to mammals with tumours in the muscular-skeletal system. In this case, survival rates of about 40% have been observed.

All this allows us to deduce, therefore, that the pharmaceutical composition or any drug prepared using it, is highly effective in the improvement of symptoms or in the prophylaxis of tumoural diseases with a carcinoma typology, which are those tumours that usually arise from a dysfunction in the regulatory systems of cell division and death in surface cells of the external and internal body, such as breast, colon or lung cancer.

In order to emphasise the surprising results of this invention, the table below shows the results obtained with other compositions and/or treatments and the survival rates obtained with the composition as described in Example 1.

TABLE 1

| Type of cancer | Composition | Survival percentage |
|---|---|---|
| Breast cancer | Squalene/omega-3/ | 100% (83% without |
| | phospholipid | recurrence and 17% with |
| | (Example 2) | a single recurrence) |
| | Squalene + tumour | 80% (All the cases |
| | removal | exhibited recurrence of |
| | | the tumour) |
| Genitourinary | Squalene/omega-3/ | 75% (There were no |
| system | phospholipid | recurrences in any case) |
| cancer | (Example 1) | |
| (bladder, kidney) | | |
| | Squalene + | 40% |
| | chemotherapy and/or | |
| | tumour removal. | |
| Colon cancer | Squalene/omega-3/ | 100% |
| | phospholipid | |
| | (Example 1) | |
| | Squalene | 50% ⁽¹⁾ |

⁽¹⁾ Source: Carcinogenesis, vol. 19 no. 2. pp. 287-290, 1998 “Chemopreventive effect of squalene on colon cancer”.

In the case of the breast and genitourinary system cancers in Table 1, the compositions of Examples 1 and 2 were applied as the sole treatment, without the application of chemotherapy, radiotherapy or tumour removal.

Now that the pharmaceutical composition of the invention and its beneficial effects in anti-cancer therapy have been sufficiently described, said composition includes all those excipients, preservatives and additives which are known by those skilled in the art, in order to prepare the most suitable galenic formulations or fodder dross, all without going beyond the scope of the invention.

The pharmaceutical composition described in the invention, which may be formulated, for example, in oral administration capsules or drinkable formulations, is easily administered to pets or domestic animals; therefore, in addition to preventing or palliating cancer symptoms in diagnosed animals, the dosage and administration of the treatment is facilitated.

A composition such as the one described is useful in the preparation of drugs designed for the treatment of cancer, and it may also be used in the preparation of formulations which act as adjuvants in alternative cancer treatments, for example, chemotherapy or radiotherapy.

It has also been observed that the use of compositions according to the invention is highly recommendable to prevent relapses in the case of animals who have had a tumour removed or have been subject to chemotherapy and radiotherapy. Therefore, it is very advantageous to use the composition with squalene, at least one phospholipid and at least one omega-3 acid to maintain the animals' good quality of life.

Furthermore, because its active ingredients are compounds with zero toxicity, since they are compounds that may be found in some types of diets in lower proportions, the composition does not have unpleasant secondary effects, such as, for example, hair loss, kidney dysfunctions or vomiting, which are very common in conventional chemotherapy treatment.

One must highlight that, although squalene inhibits one of the steps in the cholesterol biosynthetic pathway, specifically inhibiting the 3-hydroxy-3-methylglutaryl-coenzyme (HMG CoA) enzyme, this is not harmful because squalene itself is an intermediate in the same biosynthetic pathway in stages subsequent to the inhibited stage or step.

1. A pharmaceutical composition which comprises squalene or its pharmaceutically acceptable derivatives; at least one phospholipid, its salts or pharmaceutically acceptable derivatives; and at least one omega-3 fatty acid, its salts or pharmaceutically acceptable derivatives, wherein the combination of a squalene, at least one omega-3 fatty acid and at least one phospholipid are in a weight proportion of 1:1:2 or in a weight proportion of 3:5:10.

2. The pharmaceutical composition, according to claim 1, wherein the phospholipid is phosphatidylserine, phosphatidylcholine or a mixture of both.

3. A method for the prophylaxis, treatment and/or improvement of cancer symptoms in mammals, comprising administering to a subject an effective amount of the composition according to claim 1.

4. The method according to claim 3, wherein the cancer is breast cancer.

5. The method according to claim 3, wherein the cancer is skin cancer.

6. The method according to claim 3, wherein the cancer is digestive system cancer.

7. The method according to claim 3, wherein the cancer is genitourinary system cancer.

8. A method for the treatment and/or improvement of cancer in dogs and cats, comprising administering to a subject an effective amount of the composition according to claim 1.

9. The method according to claim 3, wherein the composition is administered orally.

10. The method according to claim 3, wherein the composition is administered by injection.

11. The method according to claim 3, wherein the composition is administered topically.

12. A method for the prophylaxis, treatment and/or improvement of cancer symptoms in mammals comprising administering to a subject fodder for animals which comprises an effective amount of the composition according to claim 1.

13. Fodder for animals which comprises the composition according to claim 1.

14. A pharmaceutical capsule for oral administration comprising squalene or its pharmaceutically acceptable derivatives; at least one phospholipid, its salts or pharmaceutically acceptable derivatives; and at least one omega-3 fatty acid, its salts or pharmaceutically acceptable derivatives, wherein the squalene, the omega-3 fatty acid and the phospholipid are in a weight proportion of 3:5:10.

15. A pharmaceutical suspension for oral administration which comprises squalene or its pharmaceutically acceptable derivatives; at least one phospholipid, its salts or pharmaceutically acceptable derivatives; and at least one omega-3 fatty acid, its salts or pharmaceutically acceptable derivatives, wherein the squalene, the omega-3 fatty acid and the phospholipid are in a weight proportion of 1:1:2.
